(19) United States
(12) Patent Application Publication
(10) Pub. No.: US 2023/0064082 A1
(43) Pub. Date: Mar. 2, 2023

(54) PIGTAIL DILATOR SYSTEM

(71) Applicant: Merit Medical System, Inc., South Jordan, UT (US)

(72) Inventors: Albert Sun, South Jordan, UT (US); Jason Koontz, South Jordan, UT (US); Jim Mottola, West Jordan, UT (US); Stephanie Chard, Salt Lake City, UT (US)

(21) Appl. No.: 17/822,015

(22) Filed: Aug. 24, 2022

Related U.S. Application Data
(60) Provisional application No. 63/237,919, filed on Aug. 27, 2021.
(60) Provisional application No. 63/368,181, filed on Jul. 12, 2022.

(51) Int. Cl. A61B 17/34

(52) U.S. Cl. CPC A61B 17/3496; A61B 17/3462; A61B 18/1492; A61B 2017/3425; A61B 2017/3454

(57) ABSTRACT

A catheter delivery system is disclosed. The system includes a sheath having a bend disposed proximal to a distal end, a pigtail dilator having a loop portion in the shape of a pigtail disposed proximal to a distal end, and a straight dilator. A diameter of the loop portion is smaller than a diameter of an aortic valve and larger than a cusp of the aortic valve. The sheath and pigtail dilator are percutaneously inserted together into a blood vessel without an exchange procedure and advanced together into the left ventricle of a heart through the aortic valve without an exchange procedure.

### RELATED APPLICATIONS

This application claims priority to U.S. Provisional Application No. 63/237,919, filed on Aug. 27, 2021 and titled “Pigtail Dilator System” and U.S. Provisional Application No. 63/368,181, filed on Jul. 12, 2022 and titled “Pigtail Dilator System,” both of which are hereby incorporated by reference in their entireties.

### TECHNICAL FIELD

The present disclosure relates generally to medical devices for vascular access. More specifically, the present disclosure relates to a catheter delivery system. More specifically, the present disclosure relates to a cardiac left ventricle catheter delivery system that includes a pigtail dilator.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a top view of an embodiment of a catheter delivery system.

FIG. 2 is a top view of an embodiment of a sheath of the catheter delivery system of FIG. 1.

FIG. 3 is a side view of an embodiment of a pigtail dilator of the catheter delivery system of FIG. 1.

FIG. 3A is a side view of a distal portion of the pigtail dilator of FIG. 3.

FIG. 4 is a side view of an embodiment of a straight dilator of FIG. 1.

FIG. 5 is a side view of a proximal portion of the catheter delivery system of FIG. 1 assembled for use, wherein the pigtail dilator is disposed through the sheath and a guidewire is disposed through the pigtail dilator.

FIG. 6A is a graphical view of a distal portion of the assembled catheter delivery system of FIG. 5 inserted into a vessel over a guidewire.

FIG. 6B is a graphical view of a distal portion of the assembled catheter delivery system of FIG. 5 disposed within an aorta and positioned distal to an aortic valve.

FIG. 6C is a graphical view of the distal portion of the assembled catheter delivery system of FIG. 5 disposed within the aorta and positioned distal to the aortic valve, wherein the guidewire is removed and a distal portion of the pigtail dilator forms a loop.

FIG. 6D is a graphical view of the distal portion of the assembled catheter delivery system of FIG. 5 disposed within the left ventricle of the heart and positioned proximal to the aortic valve, wherein the guidewire is removed and a distal portion of the pigtail dilator forms a loop.

FIG. 6E is a graphical view of the distal portion of the sheath of FIG. 1 disposed within the left ventricle of the heart and positioned proximal to the aortic valve.

### DETAILED DESCRIPTION

A catheter delivery system can be used to deliver a treatment or diagnostic catheter to a chamber of a heart from a peripheral location. For example, the catheter delivery system can deliver an ablation catheter to a left ventricle of the heart from a femoral artery to accomplish a cardiac ablation procedure. In other embodiments, the catheter delivery system can be used to deliver other treatment and diagnostic catheters to any chamber of the heart from other peripheral vascular access sites, such as a femoral vein. In certain embodiments, a catheter delivery system insertion procedure includes multiple catheter guidewire, dilator, and catheter exchanges to accomplish the procedure. Each of the exchanges can increase procedure time and risk of complications, such as infection.

In some embodiments, catheter delivery systems within the scope of this disclosure includes a sheath, a pigtail dilator, and a straight dilator. The sheath may include an elongate tubular body with a bend portion positioned proximal to a distal end. A connector, which may further include a hemostasis valve and a port, is positioned at a proximal end. In some embodiments, the pigtail dilator includes an elongate tubular body with a loop positioned proximal to a distal end. The loop may have a pigtail shape. A diameter of the loop is smaller than a diameter of an aortic valve and larger than an aortic valve cusp to facilitate passage of the loop through the aortic valve without catching on and damaging the cusp. A connector including a hemostasis valve and a port may be positioned at a proximal end. The straight dilator includes an elongate tubular body with a straight portion positioned proximal to a distal end. The bodies of the pigtail dilator and the straight dilator are co-axially disposable within the body of the sheath. The stiffness of the bodies may be configured to allow the dilators and the sheath to be percutaneously inserted together, without an exchange, into a blood vessel and into the left ventricle.

In use, some catheter delivery systems within the scope of this disclosure are assembled with the pigtail dilator disposed through the sheath such that the loop portion extends beyond the distal end of the sheath. The assembly is percutaneously inserted, without an exchange, into the blood vessel over a guidewire such that the sheath and the pigtail dilator are inserted into the blood vessel together. Alternatively, in some embodiments, the straight dilator may be used for the percutaneous portion of the procedure and then exchanged with the pigtail dilator. The assembly is advanced through the blood vessel until the distal end of the pigtail dilator is positioned distal to the aortic valve. The guidewire is removed, which allows the loop portion to form the pigtail shape. The loop portion and the sheath are advanced together through the aortic valve and into the left ventricle without an exchange. The pigtail dilator is removed, allowing the bend portion to form the bend. A treatment or diagnostic catheter is delivered to the left ventricle through the sheath.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

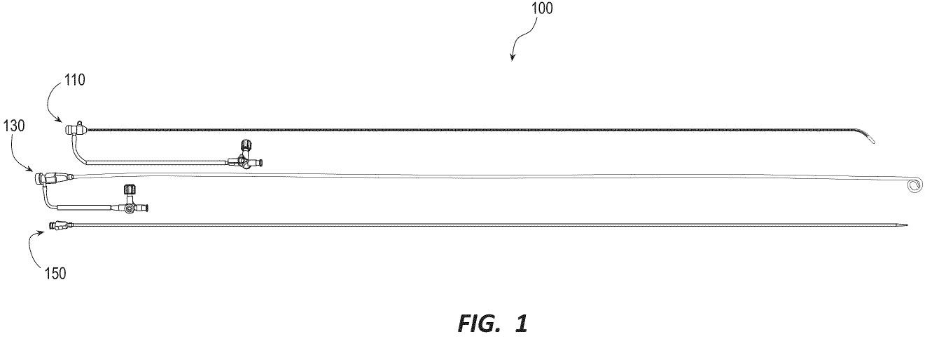

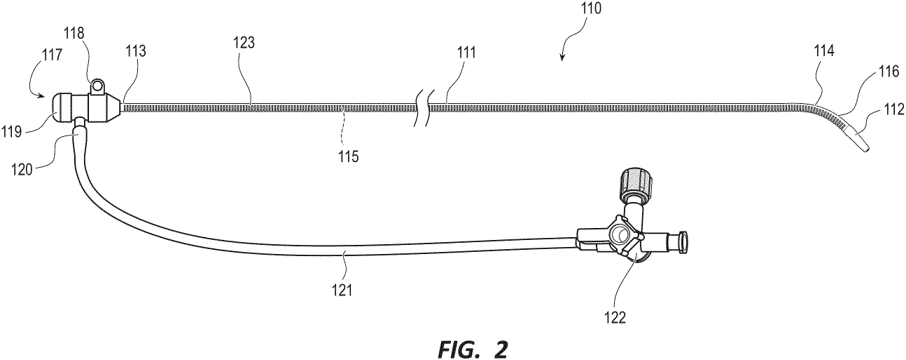

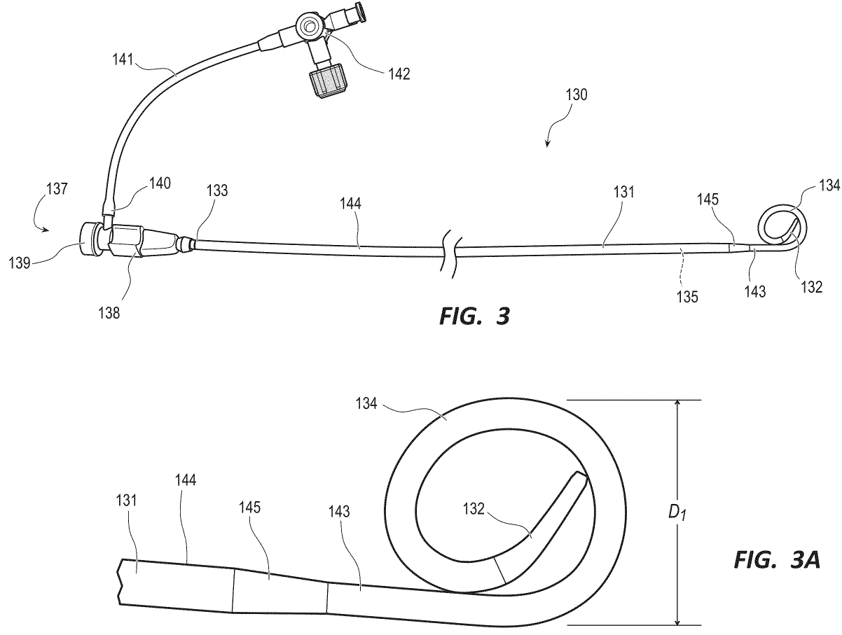

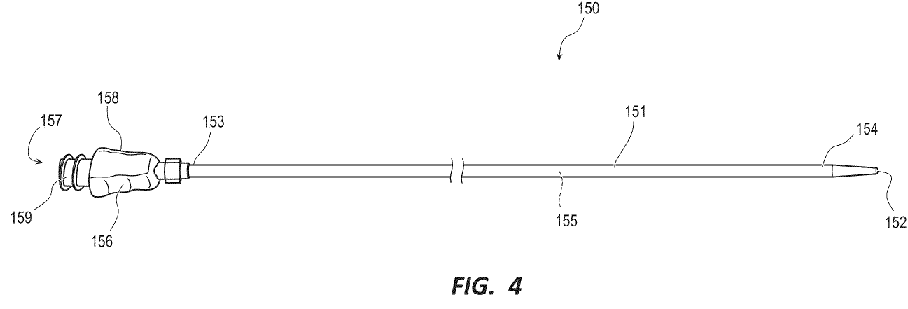

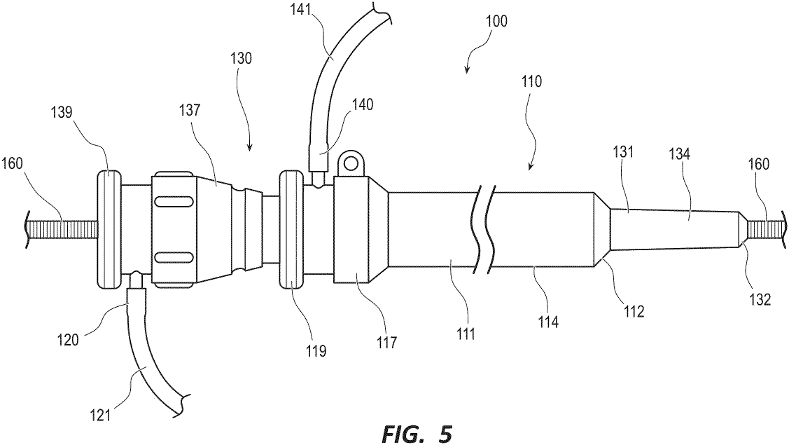

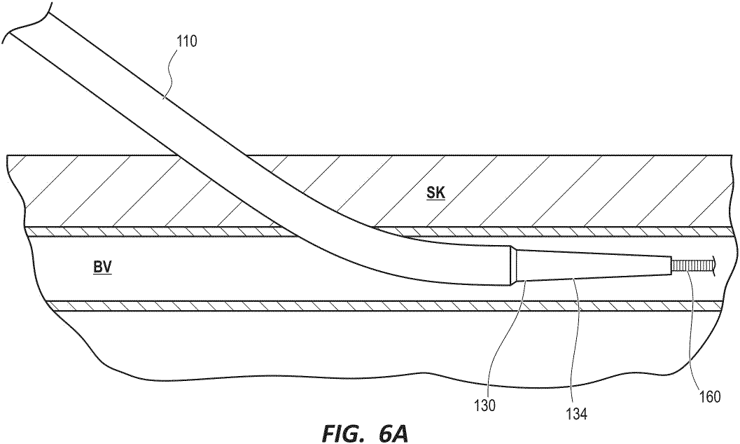

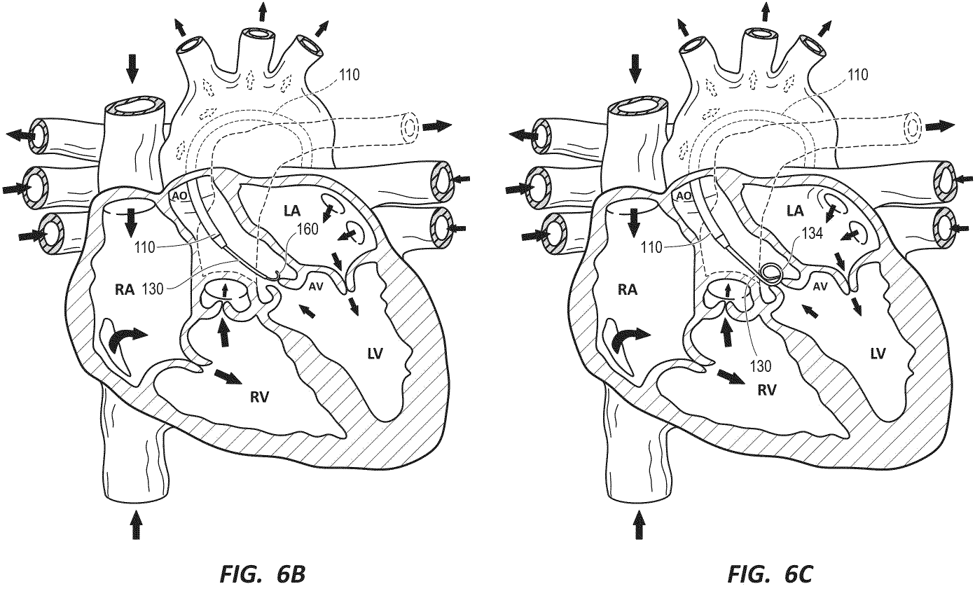

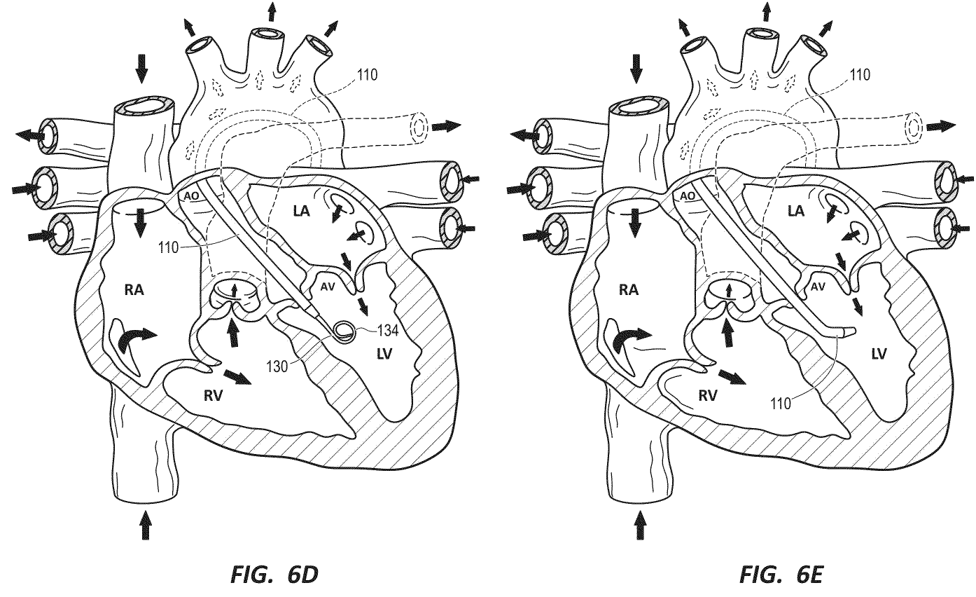

FIG. 1 illustrates an embodiment of a catheter delivery system. FIG. 2 illustrates an embodiment of a sheath of the catheter delivery system. FIGS. 3 and 3A illustrate an embodiment of a pigtail dilator of the catheter delivery system. FIG. 4 illustrates an embodiment of a straight dilator of the catheter delivery system. FIG. 5 illustrates the sheath and the pigtail dilator in an assembled state. FIGS. 6A-6E illustrate the catheter delivery system in use. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

FIG. 1 illustrates an embodiment of a catheter delivery system 100. As illustrated in FIG. 1, the catheter delivery system 100 includes a sheath 110, a first or pigtail dilator 130, and a second or straight dilator 150. In other embodiments, the catheter delivery system 100 may include additional components. For example, the catheter delivery system 100 can include a guidewire, a vascular access kit including a needle, a micro-dilator, and a guidewire introducer sheath. The catheter delivery system 100 may be used to introduce an ablation catheter through the aortic valve and into the left ventricle. In some embodiments, the catheter delivery system 100 may be configured to introduce any other suitable type of treatment catheter through a heart valve into a chamber of a patient’s heart. Notwithstanding specific examples of catheters, such as ablation catheters, recited herein, the present disclosure is applicable to a variety of catheters and elongate treatment devices, including ablation catheters, mapping catheters, guidewires, guide catheters, balloon catheters, diagnostic catheters, and so forth.

FIG. 2 illustrates the sheath 110. As illustrated, the sheath 110 comprises an elongate tubular body 111 having a distal end 112 and a proximal end 113. A lumen 115 extends through the body 111 from the distal end 112 to the proximal end 113. In some embodiments, the lumen 115 may have a diameter ranging from about 0 French to about 12 French, including diameters from about 4 French to about 10 French, and may be about 8 French or about 8.5 French, wherein 1 French is equivalent to one-third of a millimeter. In some embodiments, the outside diameter of the body may range from about 2 French to about 14 French, including diameters of about 10.5 French and may have a length from about 70 centimeters to about 150 centimeters, including lengths of about 90 centimeters. The body 111 may be formed from any suitable material, such as polyurethane, polyether block amide, polyamide 12, nylon, polypropylene, polyethylene, and polycarbonate polyurethane. Other materials are contemplated. A length of the body 111 can range from about 50 centimeters to about 130 centimeters and may be about 90 centimeters. A reinforcement member 123 may be embedded in a wall of the body 111 and configured to provide kink resistance and longitudinal stiffness to the body 111. In some embodiments, the reinforcement member 123 may be a coiled wire. The body 111 includes a bend portion 114 disposed proximal to the distal end 112. The bend portion 114 can be pre-formed during manufacturing and include an angle ranging from about 10 degrees to about 90 degrees, from about 30 degrees to about 55 degrees, and can be about 50 degrees.

A radiopaque marker 116 may be disposed proximal to the distal end 112. In some embodiments, the radiopaque marker 116 may include a radiopaque material such as barium sulfate or bismuth trioxide. Other materials are contemplated. In other embodiments, the radiopaque marker 116 may include a band formed from a material such as gold or titanium. Other materials are contemplated. The radiopaque marker 116 may facilitate determining a location, position, or orientation of the distal end 112 relative to anatomical landmarks using radiographic imaging techniques. For example, a location of the distal end 112 relative to an aortic valve of a heart may be determined radiographically.

A connector 117 may be disposed at the proximal end 113 of the body 111. The connector 117 includes a body 118 coupled to the proximal end 113. A lumen is disposed through the body 118 and is in communication with the lumen 115 of the body 111. The body 118 may include a color for indication of a size or diameter of the body 111. A hemostasis valve 119 is disposed at a proximal end of the body 118. The hemostasis valve 119 can be configured to selectively close the lumen of the body 118 to prevent blood from leaking from the sheath 110 and/or to prevent air from entering the sheath 110. The illustrated embodiment of the connector 117 includes a port 120. In other embodiments, the port 120 may not be present. The port 120 is in communication with the lumen of the body 118. An extension tube 121 is coupled to the port 120 at a distal end of the extension tube 121. An adaptor 122 is coupled to the extension tube 121 at a proximal end of the extension tube 121. In some embodiments, the adaptor 122 can be a stopcock. In other embodiments, the adaptor 122 can be a straight adaptor. The extension tube 121 and the adaptor 122 may be used to inject and/or withdraw fluid into/from the sheath 110.

FIG. 3 illustrates the pigtail dilator 130. As illustrated, the pigtail dilator 130 comprises an elongate tubular body 131 having a distal portion 143 proximal to a distal end 132 and a proximal portion 144 distal to a proximal end 133. A lumen 135 extends through the body 111 from the distal end 132 to the proximal end 133. The lumen 135 can be sized to slidingly receive a guidewire. The distal end 132 is configured to tightly surround the guidewire without a gap when the guidewire is disposed through the lumen 135. This configuration may facilitate passage of the distal end 132 through tissue (e.g., skin) without catching on the tissue. The body 131 may be formed from any suitable material, such as polyurethane, polyether block amide, polyamide 12, nylon, polypropylene, polyethylene, and polycarbonate polyurethane. Other materials are contemplated. The material of the body 131 can have a durometer hardness ranging from 40 D to 74 D, from 55 D to 63 D, and may be 58 D, per a Shore durometer type D scale. The hardness of the material can provide adequate stiffness to the body 131 to facilitate percutaneous insertion of the pigtail dilator 130 over a guidewire without longitudinal bunching of the body 131.

A radiopaque material, such as barium sulfate or bismuth trioxide, may be distributed throughout a wall of the body 131. Other materials are contemplated. The radiopaque material may facilitate determining a location, position, or orientation of the distal end 132 relative to anatomical landmarks using radiographic imaging techniques. For example, a location of the distal end 132 relative to the aortic valve of the heart may be determined radiographically. The body 131 can be sized to be slidingly disposed within the lumen 115 of the sheath 110. An outer diameter of the proximal portion 144 of the body 131 may range from about 2 French to about 14 French, including from about 4 French to about 10 French or may be from about 8 French or about 8.5 French. An outer diameter of the distal portion 143 may range from about 2 French to about 8 French, including from about 4 French to about 6.5 French, and may be about 6 French. A length of the body 131 can range from about 60 centimeters to about 160 centimeters, including from about 76 centimeter to about 156 centimeters, and may be about 100 centimeters or may be about 96 centimeters. The pigtail dilator 130 may be sized to maintain flexibility along its length and may be configured as about 6 centimeters longer than the length of the sheath 110.

The body 131 includes a loop portion 134 disposed proximal to the distal end 132. A taper 145 defines a transition from the proximal portion 143 to the distal portion 144 and provides increased flexibility to the loop portion 134. The taper 145 is disposed proximal to the loop portion 134. The loop portion 134 may be configured to facilitate passage of the pigtail dilator 130 from a position distal to the aortic valve, through the aortic valve, and into the left ventricle without catching on a cusp of the aortic valve. The loop portion 134 may include a pigtail shape. The loop portion 134 can be pre-formed during manufacturing and include an arc ranging from about 180 degrees to about 720 degrees, from about 300 degrees to about 500 degrees, and can be about 422 degrees. As shown in FIG. 3A, the loop portion 134 can include a diameter D₁ ranging from 10 millimeters to 25 millimeters and may be 16.5 millimeters. The diameter D₁ of the loop portion 134 is configured to be less than a diameter of the aortic valve such that the loop portion 134 can pass through the aortic valve without resistance. Further, the diameter D₁ is configured to be larger than the cusp of the aortic valve, such that the loop portion 134 is prevented from being caught within the cusp and resulting in an inability to pass the loop portion 134 through the aortic valve. In some embodiments, an outer diameter of the loop portion 134 can distally taper.

A connector 137 is disposed at the proximal end 133 of the body 131. The connector 137 includes a body 138 coupled to the proximal end 133. A lumen is disposed through the body 138 and is in communication with the lumen 135 of the body 131. The body 138 may include a color for indication of a size or diameter of the body 131. A hemostasis valve 139 is disposed at a proximal end of the body 138. The hemostasis valve 139 can be configured to selectively close the lumen of the body 138 to prevent blood from leaking from the pigtail dilator 130 and/or to prevent air from entering the pigtail dilator 130. The illustrated embodiment of the connector 137 includes a port 140. In other embodiments, the port 140 may not be present. The port 140 is in communication with the lumen of the body 138. An extension tube 141 is coupled to the port 140 at a distal end. An adaptor 142 is coupled to the extension tube 141 at a proximal end. In some embodiments, the adaptor 142 can be a stopcock. In other embodiments, the adaptor 142 can be a straight adaptor. The extension tube 141 and the adaptor 142 may be used to inject into and/or withdraw fluid from the pigtail dilator 130.

FIG. 4 illustrates the straight dilator 150. As illustrated, the straight dilator 150 comprises an elongate tubular body 151 having a distal end 152 and a proximal end 153. A lumen 155 extends through the body 111 from the distal end 152 to the proximal end 153. The lumen 155 can be sized to slidingly receive the guidewire. The distal end 152 is configured to tightly surround the guidewire without a gap when the guidewire is disposed through the lumen 155. This configuration may facilitate passage of the distal end 152 through tissue (e.g., skin) without catching on the tissue. The body 151 may be formed from any suitable material such as polyurethane, polyether block amide, polyamide 12, nylon, polypropylene, polyethylene, and polycarbonate polyurethane. Other materials are contemplated. The material of the body 151 can have a durometer hardness ranging from 40 D to 74 D, and may be 55 D, per the Shore durometer type D scale. The hardness of the material can provide adequate stiffness to the body 151 to facilitate percutaneous insertion of the straight dilator 150 over a guidewire without longitudinal bunching of the body 151.

A radiopaque material, such as barium sulfate or bismuth trioxide, may be distributed throughout a wall of the body 151. Other materials are contemplated. The radiopaque material may facilitate determining a location, position, or orientation of the distal end 152 relative to anatomical landmarks using radiographic imaging techniques. For example, a location of the distal end 152 relative to the aortic valve of the heart may be determined radiographically. The body 151 can be sized to be slidingly disposed within the lumen 115 of the sheath 110. An outer diameter of the body 151 may range from about 4 French to about 6.5 French and may be about 6 French. A length of the body 151 can range from about 60 centimeters to about 160 centimeters, including from about 76 centimeters to about 156 centimeters, and may be about 96 centimeters. The body 151 includes a straight portion 154 disposed proximal to the distal end 152. The straight portion 154 may be configured to facilitate percutaneous insertion of the straight dilator 150 into the blood vessel. In some embodiments, an outer diameter of the straight portion 154 can distally taper from about 8 French to about 6 French.

In the illustrated embodiment, a connector 157 is disposed at the proximal end 153 of the body 151. The connector 157 includes a body 158 coupled to the proximal end 153. A lumen is disposed through the body 158 and is in communication with the lumen 155 of the body 151. The body 158 may include a color for indication of a size or diameter of the body 151. In the depicted embodiment, a female Luer fitting 159 is disposed at a proximal end of the body 158. The female Luer fitting 159 may be configured to couple with a male Luer fitting of a medical device (e.g., syringe). In other embodiments, the connector 157 may include a hemostasis valve. In still other embodiments, the connector 157 can include an extension tube coupled to a port and an adaptor.

Notwithstanding specific examples given above, the durometers, length, reinforcement, and other features the sheath 110, the pigtail dilator 130, and/or the straight dilator 150 may vary in various embodiments within the scope of this disclosure. For example, the sheath 110, the pigtail dilator 130, and/or the straight dilator 150 may or may not include braids or other reinforcement members in the wall of the device to reinforce, strengthen, enhance torquability, or impart other properties to the component. Similarly, the sheath 110, the pigtail dilator 130, and/or the straight dilator 150 may or may not include a hydrophilic coating. Other variations to these components are likewise within the scope of this disclosure.

FIG. 5 illustrates the catheter delivery system 100 assembled in a ready state and disposed over a guidewire 160. As illustrated, the sheath 110 is disposed over the pigtail dilator 130 and the pigtail dilator 130 is disposed over the guidewire 160. The body 131 is disposed through the hemostasis valve 119 of the connector 117 and extends distally from the distal end 112 of the body 111. The distal end 112 tightly surrounds the body 131 without gaps as previously discussed. The bend portion 114 of the body 111 is shown in a straightened configuration. The bend portion 114 may be straightened by the body 131. The loop portion 134 of the body 131 extends from the distal end 112 and is shown in a straightened configuration. The loop portion 134 can be straightened by the guidewire 160. The guidewire 160 is disposed through the hemostasis valve 139 of the connector 137 and extends from the distal end 132. The distal end 132 tightly surrounds the guidewire 160 as previously discussed. In other embodiments, the straight dilator 150 may be assembled to the sheath 110 similarly as the pigtail dilator 130 is assembled to the sheath 110 as shown in FIG. 5. In other words, the pigtail dilator 130 and the straight dilator 150 may be interchangeable.

FIGS. 6A-6B illustrate the catheter delivery system 100 in use. As illustrated in FIG. 6A, the guidewire 160 is percutaneously inserted into a blood vessel BV. The pigtail dilator 130 is disposed over the guidewire 160 and the sheath 110 is disposed over the pigtail dilator 130 such that the pigtail dilator 130 and the sheath 110 are inserted together into the blood vessel BV. In another embodiment, the straight dilator 150 may be inserted together with the sheath 110 into the blood vessel BV. Once inserted, the straight dilator 150 may be interchanged with the pigtail dilator 130.

As illustrated in FIG. 6B, a distal end of the guidewire 160 is positioned distal to an aortic valve AV and distal portions of the pigtail dilator 130 and the sheath 110 are disposed within an aorta AO. The positioning of the guidewire 160 and the distal portions of the pigtail dilator 130 and the sheath 110 may be achieved with utilization of a radiographic imaging system.

As illustrated in FIG. 6C, the guidewire 160 is removed and the loop portion 134 of the pigtail dilator 130 is allowed to form the pigtail shape within the aorta AO and distal to the aortic valve AV. As illustrated in FIG. 6D, the loop portion 134 is passed through the cusps of the aortic valve AV such that the pigtail dilator 130 and the sheath 110 are advanced together into the left ventricle LV. As illustrated in FIG. 6E, the pigtail dilator 130 is removed and the bend portion 114 is allowed to reform a bend.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of inserting a catheter delivery system into a left ventricle of a heart may include one or more of the following steps: inserting a dilator and a sheath together over a guidewire into a blood vessel, wherein the dilator is co-axially disposed within the sheath; positioning a distal portion of the dilator distal to an aortic valve of the heart, wherein the distal portion is straight; allowing the distal portion of the dilator to form a loop; advancing the dilator and the sheath together proximally past cusps of the aortic valve into the left ventricle, wherein a distal portion of the sheath and the distal portion of the dilator are disposed within the left ventricle of the heart; and removing the dilator from the sheath. Other steps are also contemplated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use.

“Fluid” is used in its broadest sense, to refer to any fluid, including both liquids and gases as well as solutions, compounds, suspensions, etc., which generally behave as fluids.

References to approximations are made throughout this specification, such as by use of the term “about.” For each such reference, it is to be understood that, in some embodiments, the value, feature, or characteristic may be specified without approximation. For example, where a qualifier such as “about” is used, this term includes within its scope the qualified words in the absence of its qualifier.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A catheter delivery system, comprising:
a sheath comprising a sheath body comprising:
a distal end;
a proximal end;
a bend portion disposed proximal to the distal end; and
a sheath lumen extending through the sheath body from the distal end to the proximal end of the sheath body; and
a dilator comprising a dilator body comprising:
a distal end:
a proximal end; and
a loop portion disposed proximal to the distal end of the dilator body,
wherein the dilator body is co-axially disposable within the sheath lumen, and
wherein the loop portion extends from the distal end of the sheath body when the dilator body is disposed within the sheath lumen;
wherein the sheath and the dilator are configured to be percutaneously inserted into a blood vessel together; and
wherein the sheath and dilator are configured to be passed into a cardiac left ventricle together.

2. The system of claim 1, wherein the loop portion comprises an arc ranging from 180 degrees to 720 degrees.

3. The system of claim 1, wherein the loop portion comprises a diameter ranging from 10 millimeters to 25 millimeters.

4. The system of claim 3, wherein the diameter of the loop portion is smaller than a diameter of an aortic valve.

5. The system of claim 3, wherein the diameter of the loop portion is larger than a cusp of an aortic valve.

6. The system of claim 1, wherein a diameter of the dilator body within the loop portion tapers distally from an 8 French diameter to a 6 French diameter.

7. The system of claim 1, wherein the dilator body comprises a material having a durometer hardness ranging from 40 Shore D to 74 Shore D.

8. The system of claim 1, wherein the dilator further comprises a dilator connector coupled to the proximal end of the dilator body, comprising:
a dilator connector body coupled to the proximal end of the dilator body comprising a dilator connector lumen in communication with a dilator body lumen;
a dilator hemostasis valve disposed at a proximal end of the dilator connector body configured to selectively close the dilator connector lumen; and
a dilator connector port in communication with the dilator connector lumen.

9. The system of claim 8, wherein the dilator connector further comprises:
a dilator connector tube coupled to the dilator connector port at a distal end; and
a dilator connector adaptor coupled to a proximal end of the dilator connector tube,
wherein the dilator connector adaptor is in communication with the dilator connector lumen.

10. The system of claim 1, further comprising a guidewire co-axially disposable within a dilator body lumen and wherein the distal end of the dilator body is configured to fit tightly, without a gap, around the guidewire, wherein the distal end is configured to pass through tissue without catching on the tissue.

11. The system of claim 1, wherein the bend portion of the sheath body comprises an angle ranging from 10 degrees to 90 degrees.

12. A method of inserting a catheter delivery system into a left ventricle of a heart, comprising:
inserting a dilator and a sheath together over a guidewire into a blood vessel, wherein the dilator is co-axially disposed within the sheath;
positioning a distal portion of the dilator distal to an aortic valve of the heart, wherein the distal portion is straight;
allowing the distal portion of the dilator to form a loop;
advancing the dilator and the sheath together past cusps of the aortic valve into the left ventricle, wherein a distal portion of the sheath and the distal portion of the dilator are disposed within the left ventricle of the heart; and
removing the dilator from the sheath.

13. The method of claim 12, further comprising:
percutaneously inserting the guidewire into the blood vessel; and
positioning a distal end of the guidewire distal to the aortic valve.

14. The method of claim 12, wherein allowing the distal portion of the dilator to form a loop comprises removing the guidewire from a lumen of the dilator.

15. The method of claim 14, wherein a diameter of the loop is smaller than the aortic valve.

16. The method of claim 14, wherein a diameter of the loop is larger than the cusps of the aortic valve.

17. A catheter delivery system kit, comprising:
a sheath comprising a sheath body comprising:
a bend portion disposed proximal to a distal end; and
a sheath lumen extending through the sheath body from the distal end to a proximal end of the sheath body;
a first dilator comprising a first dilator body comprising:
a loop portion disposed proximal to a distal end,
wherein the dilator body is co-axially disposable within the sheath lumen, and
wherein the loop portion extends from the distal end of the sheath body when the dilator body is disposed within the sheath lumen;
wherein the sheath and the first dilator are configured to be percutaneously inserted into a blood vessel together; and
wherein the sheath and first dilator are configured to be passed into a cardiac left ventricle together.

18. The kit of claim 17, further comprising a second dilator comprising:
a second dilator body comprising a straight portion disposed proximal to a distal end,
wherein the second dilator body is co-axially disposable within the sheath lumen, and
wherein the straight portion extends from the distal end of the sheath body when the second dilator body is disposed within the sheath lumen.

19. The kit of claim 18, further comprising a guidewire disposable within a lumen of the first dilator and a lumen of the second dilator.

20. The kit of claim 17, further comprising a hemostasis valve disposed at a proximal end of the first dilator.
